(19) United States
(12) Patent Application Publication
(10) Pub. No.: US 2024/0100217 A1
(43) Pub. Date: Mar. 28, 2024

(54) KAOLIN-BASED HEMOSTATIC GAUZE AND PREPARATION METHOD THEREOF

(71) Applicant: CHINA UNIVERSITY OF GEOSCIENCES (WUHAN), Wuhan (CN)

(72) Inventors: Huaming YANG, Wuhan (CN); Qiying ZHENG, Wuhan (CN); Yunyang LIU, Wuhan (CN); Juan LIAO, Wuhan (CN)

(73) Assignee: CHINA UNIVERSITY OF GEOSCIENCES (WUHAN), Wuhan (CN)

(21) Appl. No.: 18/472,258

(22) Filed: Sep. 22, 2023

(30) Foreign Application Priority Data
Sep. 27, 2022 (CN) 202211182898.X

(51) Int. Cl. A61L 15/44; A61L 15/18; C04B 33/04; C04B 33/13; C04B 33/32

(52) U.S. Cl. CPC A61L 15/44; A61L 15/18; C04B 33/04; C04B 33/131; C04B 33/326; A61L 2300/102; A61L 2300/404; A61L 2300/418; C04B 2235/349

(57) ABSTRACT

A kaolin-based hemostatic gauze comprises: a medical non-woven fabric as a carrier; and a kaolin-containing composite hemostatic material loaded on the medical non-woven fabric, wherein kaolin serves as a carrier in the kaolin-containing composite hemostatic material, the kaolin is doped with Ce and α-Fe₂O₃ is loaded on the Kaolin. A method for preparing the kaolin-based hemostatic gauze, includes: S1: preparing the kaolin-containing composite hemostatic material; S2: preparing the kaolin-containing composite hemostatic material into a suspension; S3: impregnating the medical non-woven fabric with the suspension thoroughly stirred, wherein upper and lower sides of the medical non-woven fabric each are impregnated once; and S4: pressing and oven-drying an impregnated medical non-woven fabric to obtain the kaolin-based hemostatic gauze. In the method, the medical non-woven fabric is combined with the powdery hemostatic material Ce-α-Fe₂O₃/Kaol through impregnation to prepare the medical hemostatic gauze product with excellent hemostatic performance, prominent biocompatibility, high safety, and antibacterial activity.

### CROSS-REFERENCE TO RELATED APPLICATIONS

This application claims priority to Chinese Patent Application No. 202211182898.X with a filing date of Sep. 27, 2022. The content of the aforementioned application, including any intervening amendments thereto, is incorporated herein by reference.

### TECHNICAL FIELD

The present disclosure relates to the technical field of hemostatic gauzes, and in particular to a kaolin-based hemostatic gauze and a preparation method thereof.

### BACKGROUND

The hemostatic products currently available on the market mainly include adhesive bandages, medical hemostatic gauzes, medical hemostatic bandages, etc. Common hemostatic dressings are spun from cotton, non-woven fabrics, etc. These hemostatic dressings themselves have certain hemostatic effect. However, the hemostatic effect is limited. It is a hot spot in the field of biological hemostasis to combine a hemostatic material with a gauze to prepare a multi-functional hemostatic dressing. The current hemostatic dressings such as chitosan-based dressings on the market are cost-ineffective. Inorganic materials such as natural clay have been reported to have excellent hemostatic effects. For example, in a Quikclot kaolinite-based hemostatic bandage produced by Z-Medica in the United States, a kaolinite powder is successfully loaded on a gauze, and the bandage has been confirmed to have excellent hemostatic performance. However, commercially-available hemostatic agents still have shortcomings such as single components and limited functions, which restrict the development and application of high-performance hemostatic products.

### SUMMARY OF PRESENT INVENTION

An objective of the present disclosure is to provide a kaolin-based hemostatic gauze with excellent hemostatic performance, prominent biocompatibility, high safety, and antibacterial activity. A preparation method thereof is further provided.

The kaolin-containing composite hemostatic material comprises a medical non-woven fabric as a carrier; and a kaolin-containing composite hemostatic material load on the medical non-woven fabric. Kaolin serves as a carrier in the kaolin-containing composite hemostatic material, the Kaolin is doped with Ce, and α-Fe₂O₃ is loaded on the Kaolin.

In one embodiment, the kaolin includes flaky kaolinite and tubular halloysite.

In one embodiment, the kaolin is a raw ore.

In one embodiment, a loading rate of the α-Fe₂O₃ is 50% to 70%.

In one embodiment, the kaolin-containing composite hemostatic material is prepared according to the following steps:
S1: mechanically mixing the kaolin with a polyhydroxyferric ion solution and a Ce salt solution; and
S2: calcining a resulting mixture at a specified temperature to obtain a hemostatic material α-Fe₂O₃/Kaol with high biocompatibility, namely, the kaolin-containing composite hemostatic material.

In one embodiment, the calcination is conducted at 500° C. to 600° C.

In one embodiment, the calcination is conducted at 550° C.

In one embodiment, the polyhydroxyferric ion solution has a concentration of 0.4 mol/L, and a mass-to-volume ratio of the kaolin to the polyhydroxyferric ion solution is 1:50 g/mL; the Ce salt solution is a Ce(NO₃)₃ solution; and the Ce(NO₃)₃ solution has a concentration of 0.1 mol/L to 1.0 mol/L.

The method for preparing the kaolin-based hemostatic gauze, includes the following steps:
S1: preparing the kaolin-containing composite hemostatic material;
S2: preparing the kaolin-containing composite hemostatic material into a suspension;
S3: impregnating the medical non-woven fabric with the suspension thoroughly stirred, wherein upper and lower sides of the medical non-woven fabric each are impregnated once; and
S4: pressing and oven-drying an impregnated medical non-woven fabric to obtain the kaolin-based hemostatic gauze.

In one embodiment, the suspension of the kaolin-containing composite hemostatic material has a concentration of 0.001 g/mL to 0.05 g/mL.

In the kaolin-containing composite hemostatic material according to the present disclosure, α-Fe₂O₃ is loaded and Ce is doped to enhance a hemostatic effect; and the prepared Ce-α-Fe₂O₃/Kaol hemostatic material has no obvious cytotoxicity, no hemolysis, excellent biocompatibility, high safety, and antibacterial activity. Moreover, the preparation method involves simple steps and easy operations, and is conducive to large-scale production.

According to embodiments of the present disclosure, the medical non-woven fabric is combined with the powdery hemostatic material Ce-α-Fe₂O₃/Kaol through impregnation to prepare a medical hemostatic gauze product with excellent hemostatic performance, prominent biocompatibility, high safety, and antibacterial activity. The preparation method thereof is simple and is conducive to industrial production.

### BRIEF DESCRIPTION OF THE DRAWINGS

FIG. 1 shows morphological images of flaky kaolinite, tubular halloysite, and kaolin;

FIG. 2 shows test results of cytotoxicity of the α-Fe₂O₃/Kaol composite hemostatic material and α-Fe₂O₃ prepared in each of Examples 1 to 5 of the present disclosure;

FIG. 3 shows test results of hemolysis of the α-Fe₂O₃/Kaol composite hemostatic material and α-Fe₂O₃ prepared in each of Examples 1 to 5 of the present disclosure;

FIG. 4 shows test results of in vitro procoagulant effects of kaolin and different iron oxide/Kaol composite hemostatic materials;

FIG. 5 shows test results of in vitro bleeding times of the Ce-α-Fe₂O₃/Kaol composite hemostatic materials prepared in Examples 7 to 9, a raw ore, the α-Fe₂O₃/Kaol prepared in Example 6, and the control group;

FIG. 6 shows test results of hemolysis of flaky kaolinite, tubular halloysite, and kaolin;

FIG. 7 is a schematic diagram showing the method for preparing the hemostatic gauze according to one embodiment of the present disclosure;

FIG. 8 shows pictures of 6 gauze products with different loads obtained through impregnation with hemostatic material suspensions of different concentrations (Examples 10 to 15);

FIG. 9 shows experimental results of liver hemostasis in animals with the Ce-α-Fe₂O₃/Kaol hemostatic gauze prepared in an embodiment of the present disclosure, an ordinary gauze, a Quikclot gauze (with kaolinite as a main component), and a Ce-α-Fe₂O₃/Kaol hemostatic powder; and

FIG. 10 shows experimental results of tail vein hemostasis in animals with the Ce-α-Fe₂O₃/Kaol hemostatic gauze prepared in an embodiment of the present disclosure, an ordinary gauze, a Quikclot gauze (with kaolinite as a main component), and a Ce-α-Fe₂O₃/Kaol hemostatic powder.

### DETAILED DESCRIPTION OF THE EMBODIMENTS

The technical solutions of the present disclosure are described in further detail below with reference to the specific embodiments and accompanying drawings, but the present disclosure is not limited thereto.

The term “kaolin” in the disclosure has a chemical formula of Al₂O₃·2SiO₂·2H₂O; and in some cases, the kaolin includes about 45.31% of silica, about 37.21% of alumina, and about 14.1% of water.

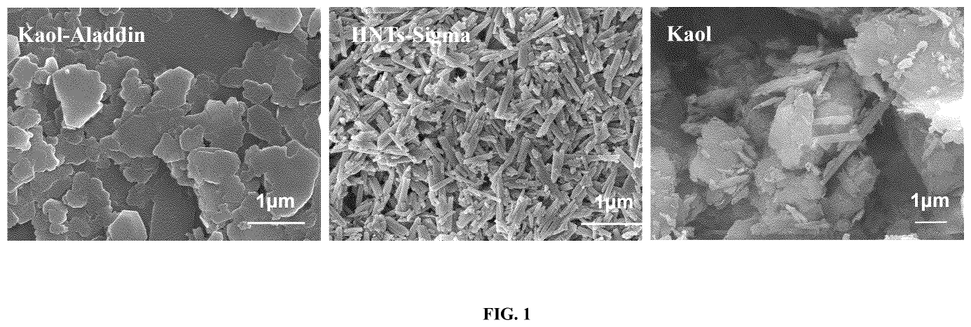

It has been found through quantitative mineralogical analysis by X-ray diffraction (XRD) that the kaolin used in the embodiments of the disclosure includes halloysite and kaolinite; and it is found that the halloysite is tubular and the kaolinite is flaky according to scanning electron microscopy (SEM) analysis results shown in FIG. 1.

### Preparation of Kaolin

A method for preparing a kaolin sample includes the following steps: crushing kaolin raw ores with a crusher to obtain a powder, mixing the powder thoroughly by a nine-square grid beneficiation method, and collecting a material in a middle of a nine-square grid and further grinding with a three-head grinder to obtain the sample.

### Preparation of a Polyhydroxyferric Ion Solution

A polyhydroxyferric ion solution with a concentration of 0.4 mol/L is prepared with FeCl₃·6H₂O and NaOH.

### Preparation of a Ce(NO₃)₃ Solution

A 0.12 mol/L Ce(NO₃)₃ solution is prepared with Ce(NO₃)₃·6H₂O and water.

### Preparation of α-Fe₂O₃/Kaol Composite Products with Different Loading Rates (Examples 1 to 6)

### Example 1

In this example, a preparation method of a α-Fe₂O₃/Kaol₁ composite hemostatic material with a α-Fe₂O₃ content of 50.41% was provided. The preparation method included the following steps: 5 g of the Kaolin sample and 250 mL of the polyhydroxyferric ion solution were mixed, a pH of a resulting mixture was adjusted with a 5 mol/L NaOH solution to about 3, a resulting system was stirred at 60° C. for 5 h and then centrifuged at 8,000 rpm, and a resulting precipitate was washed 3 times and dried to obtain a FeOOH/Kaol kaolin composite. The FeOOH/Kaol kaolin composite was ground and then calcined (including calcining at 250° C. for 1 h, calcining at 350° C. for 1 h, and calcining at 550° C. for 4 h) to obtain the α-Fe₂O₃/Kaol₁ composite hemostatic material with a α-Fe₂O₃ content of 50.41%.

### Example 2

In this example, a preparation method of a α-Fe₂O₃/Kaol₂ composite hemostatic material with a α-Fe₂O₃ content of 34.52% was provided. The preparation method included the following steps: 10 g of the Kaolin sample and 250 mL of the polyhydroxyferric ion solution were mixed, a pH of a resulting mixture was adjusted with a 5 mol/L NaOH solution to about 3, a resulting system was stirred at 60° C. for 5 h and then centrifuged at 8,000 rpm, and a resulting precipitate was washed 3 times and dried to obtain a FeOOH/Kaol kaolin composite. The FeOOH/Kaol kaolin composite was ground and then calcined (including calcining at 250° C. for 1 h, calcining at 350° C. for 1 h, and calcining at 550° C. for 4 h) to obtain the α-Fe₂O₃/Kaol₂ composite hemostatic material with a α-Fe₂O₃ content of 34.52%.

### Example 3

In this example, a preparation method of a α-Fe₂O₃/Kaol₄ composite hemostatic material with a α-Fe₂O₃ content of 22.29% was provided. The preparation method included the following steps: 20 g of the Kaolin sample and 250 mL of the polyhydroxyferric ion solution were mixed, a pH of a resulting mixture was adjusted with a 5 mol/L NaOH solution to about 3, a resulting system was stirred at 60° C. for 5 h and then centrifuged at 8,000 rpm, and a resulting precipitate was washed 3 times and dried to obtain a FeOOH/Kaol kaolin composite. The FeOOH/Kaol kaolin composite was ground and then calcined (including calcining at 250° C. for 1 h, calcining at 350° C. for 1 h, and calcining at 550° C. for 4 h) to obtain the α-Fe₂O₃/Kaol₄ composite hemostatic material with a α-Fe₂O₃ content of 22.29%.

### Example 4

In this example, a preparation method of a α-Fe₂O₃/Kaol₈ composite hemostatic material with a α-Fe₂O₃ content of 6.26% was provided. The preparation method included the following steps: 40 g of the Kaolin sample and 250 mL of the polyhydroxyferric ion solution were mixed, a pH of a resulting mixture was adjusted with a 5 mol/L NaOH solution to about 3, a resulting system was stirred at 60° C. for 5 h and then centrifuged at 8,000 rpm, and a resulting precipitate was washed 3 times and dried to obtain a FeOOH/Kaol kaolin composite. The FeOOH/Kaol kaolin composite was ground and then calcined (including calcining at 250° C. for 1 h, calcining at 350° C. for 1 h, and calcining at 550° C. for 4 h) to obtain the α-Fe₂O₃/Kaol₈ composite hemostatic material with a α-Fe₂O₃ content of 6.26%.

### Example 5

In this example, a preparation method of a α-Fe₂O₃/Kaol₁₀ composite hemostatic material with a α-Fe₂O₃ content of 7.45% was provided. The preparation method included the following steps: 50 g of the Kaolin sample and 250 mL of the polyhydroxyferric ion solution were mixed, a pH of a resulting mixture was adjusted with a 5 mol/L NaOH solution to about 3, a resulting system was stirred at 60° C. for 5 h and then centrifuged at 8,000 rpm, and a resulting precipitate was washed 3 times and dried to obtain a FeOOH/Kaol kaolin composite. The FeOOH/Kaol kaolin composite was ground and then calcined (including calcining at 250° C. for 1 h, calcining at 350° C. for 1 h, and calcining at 550° C. for 4 h) to obtain the α-Fe₂O₃/Kaol₁₀ composite hemostatic material with a α-Fe₂O₃ content of 7.45%.

### Example 6

In this example, a preparation method of a α-Fe₂O₃/Kaol composite hemostatic material with a α-Fe₂O₃ content of 62.19% was provided. The preparation method included the following steps: 30 g of the Kaolin sample and 1500 mL of the polyhydroxyferric ion solution were mixed, a pH of a resulting mixture was adjusted with a 5 mol/L NaOH solution to about 3, a resulting system was stirred at 60° C. for 5 h and then centrifuged at 8,000 rpm, and a resulting precipitate was washed 3 times and dried to obtain a FeOOH/Kaol kaolin composite. The FeOOH/Kaol kaolin composite was ground and then calcined (including calcining at 250° C. for 1 h, calcining at 350° C. for 1 h, and calcining at 550° C. for 4 h) to obtain the α-Fe₂O₃/Kaol composite hemostatic material with a α-Fe₂O₃ content of 62.19%.

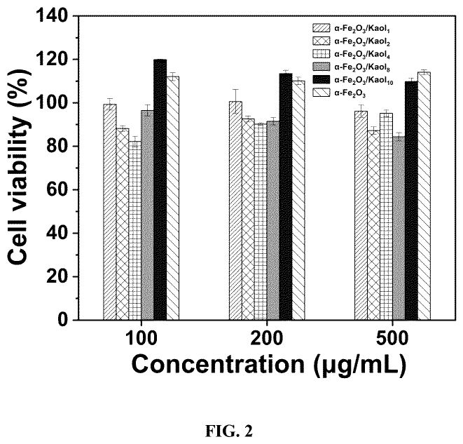

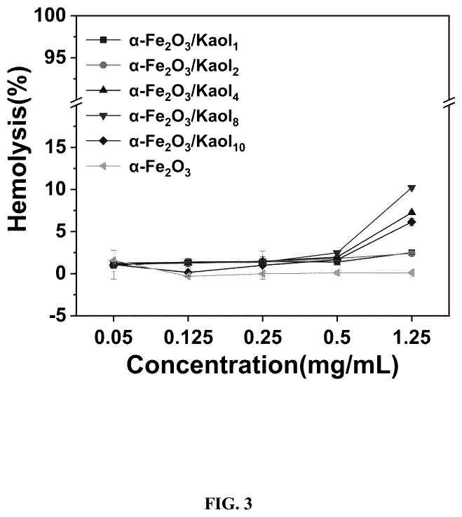

The α-Fe₂O₃/Kaol₁, α-Fe₂O₃/Kaol₂, α-Fe₂O₃/Kaol₄, α-Fe₂O₃/Kaol₈, and α-Fe₂O₃/Kaol₁₀ composite hemostatic materials and α-Fe₂O₃ prepared in Examples 1 to 5 each were subjected to a cytotoxicity test (FIG. 2) and a hemolysis test (FIG. 3). Test results showed that α-Fe₂O₃ itself has high cell viability and low hemolysis, and the α-Fe₂O₃/Kaol hemostatic material with a α-Fe₂O₃ loading rate of greater than 50% exhibits better biocompatibility than hemostatic materials with α-Fe₂O₃ loading rates of less than 50%.

### Preparation of Ce-α-Fe₂O₃/Kaol Composite Hemostatic Materials

### Example 7

In this example, a preparation method of a Ce-α-Fe₂O₃/Kaol composite hemostatic material was provided. The preparation method included the following steps: 5 g of the kaolin sample and 250 mL of the polyhydroxyferric ion solution were mixed and stirred, 20 mL of the Ce(NO₃)₃ solution was added dropwise, and a pH of a resulting mixed solution was adjusted with a 5 mol/L NaOH solution to about 3; a resulting system was stirred at 60° C. for 5 h and then centrifuged at 8,000 rpm, and a resulting precipitate was washed 3 times and dried to obtain a Ce—FeOOH/Kaol composite. The Ce—FeOOH/Kaol composite was ground and then calcined (including calcining at 250° C. for 1 h, calcining at 350° C. for 1 h, and calcining at 550° C. for 4 h) to obtain the Ce-α-Fe₂O₃/Kaol composite hemostatic material. The Ce-α-Fe₂O₃/Kaol composite hemostatic material was subjected to composition analysis by X-ray fluorescence (XRF) spectrometry (Table 1).

TABLE 1
Analysis results of oxide contents in the Ce-α-Fe₂O₃/
Kaol composite hemostatic material

| Component | Content (wt %) |
|---|---|
| Fe₂O₃ | 41.54 |
| SiO₂ | 41.29 |
| Al₂O₃ | 15.07 |
| K₂O | 1.37 |
| CeO₂ | 0.0566 |

It can be seen from Table 1 that, in the Ce-α-Fe₂O₃/Kaol, a load of Fe₂O₃ is 41.54%, and a small amount of Ce is successfully doped.

### Example 8

In this example, a preparation method of a α-Fe₂O₃—Ce/Kaol-Aladdin composite hemostatic material was provided. The preparation method included the following steps: 5 g of flaky kaolinite (Kaol-Aladdin; Aladdin, CAS:1332-58-7) and 250 mL of the polyhydroxyferric ion solution were mixed and stirred, 20 mL of the Ce(NO₃)₃ solution was added dropwise, and a pH of a resulting mixed solution was adjusted with a 5 mol/L NaOH solution to about 3; a resulting system was stirred at 60° C. for 5 h and then centrifuged at 8,000 rpm, and a resulting precipitate was washed 3 times and dried to obtain a Ce—FeOOH/Kaol-Aladdin composite. The Ce—FeOOH/Kaol-Aladdin composite was ground and then calcined (including calcining at 250° C. for 1 h, calcining at 350° C. for 1 h, and calcining at 550° C. for 4 h) to obtain the Ce-α-Fe₂O₃/Kaol-Aladdin composite hemostatic material.

### Example 9

In this example, a preparation method of a Ce-α-Fe₂O₃/HNTs-Sigma composite hemostatic material was provided. The preparation method included the following steps: 5 g of tubular halloysite (HNTs-Sigma; Sigma, CAS: 12298-43-0) and 250 mL of the polyhydroxyferric ion solution were mixed and stirred, 20 mL of the Ce(NO₃)₃ solution was added dropwise, and a pH of a resulting mixed solution was adjusted with a 5 mol/L NaOH solution to about 3; a resulting system was stirred at 60° C. for 5 h and then centrifuged at 8,000 rpm, and a resulting precipitate was washed 3 times and dried to obtain a Ce—FeOOH/HNTs-Sigma composite. The Ce—FeOOH/HNTs-Sigma composite was ground and then calcined (including calcining at 250° C. for 1 h, calcining at 350° C. for 1 h, and calcining at 550° C. for 4 h) to obtain the Ce-α-Fe₂O₃/HNTs-Sigma composite hemostatic material.

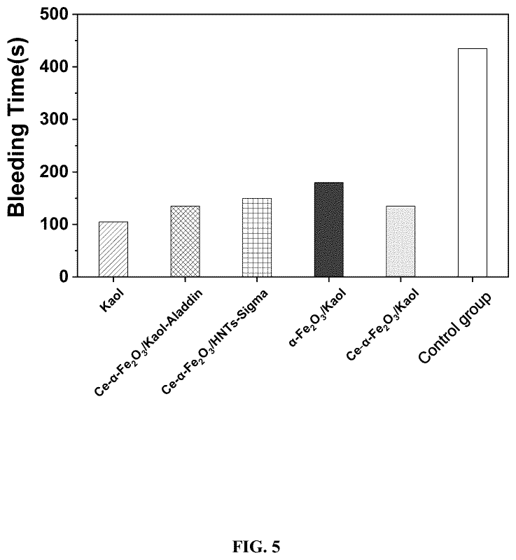

The above hemostatic materials each were subjected to an in vitro bleeding time test (FIG. 5). Test results showed that hemostatic effects of the Ce-α-Fe₂O₃/Kaol-Aladdin composite hemostatic material (Example 8) and the Ce-α-Fe₂O₃/HNTs-Sigma composite hemostatic material (Example 9) were comparable to a hemostatic effect of the Ce-α-Fe₂O₃/Kaol composite hemostatic material (Example 7), and are more significant than a hemostatic effect of the α-Fe₂O₃/Kaol (Example 6), indicating that the Ce-doped composite hemostatic materials exhibited significantly-improved hemostatic effects.

### Preparation of α-Fe₂O₃/Kaol, Fe₃O₄/Kaol, γ-Fe₂O₃/Kaol, and FeOOH/Kaol Composite Hemostatic Materials

A preparation method of the α-Fe₂O₃/Kaol, Fe₃O₄/Kaol, γ-Fe₂O₃/Kaol, and FeOOH/Kaol composite hemostatic materials was provided. The preparation method included the following steps: 5 g of a kaolin sample and 250 mL of the polyhydroxyferric ion solution were mixed, a pH of a resulting mixture was adjusted with a 5 mol/L NaOH solution to about 3, a resulting system was stirred at 60° C. for 5 h and then centrifuged at 8,000 rpm, and a resulting precipitate was washed 3 times and dried to obtain a FeOOH/Kaol composite; the FeOOH/Kaol composite was ground and then calcined (including calcining at 250° C. for 1 h, calcining at 350° C. for 1 h, and calcining at 550° C. for 4 h) to obtain the α-Fe₂O₃/Kaol composite hemostatic material; the α-Fe₂O₃/Kaol composite obtained after calcination was calcined for 1 h at 450° C. in a H₂/Ar (volume ratio: 1:9) atmosphere to obtain the Fe₃O₄/Kaol. The Fe₃O₄/Kaol was calcined for 2 h at 250° C. in an air atmosphere to obtain the γ-Fe₂O₃/Kaol.

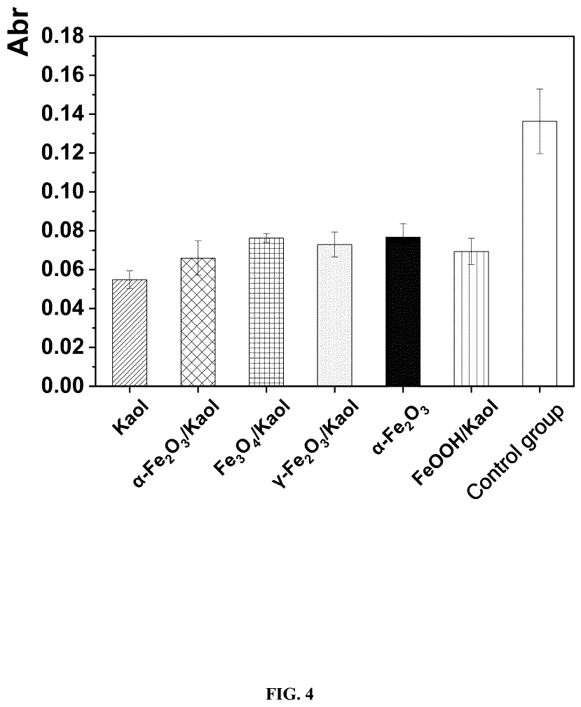

Given that different oxide types may have different impacts on improving a hemostatic effect of kaolin, the prepared α-Fe₂O₃/Kaol, Fe₃O₄/Kaol, γ-Fe₂O₃/Kaol, and FeOOH/Kaol were evaluated through an in vivo procoagulant test (FIG. 4), and evaluation results showed that α-Fe₂O₃/Kaol exhibited the optimal procoagulant performance.

Hemolysis Experiment:

Preparation of a 2% red blood cell (RBC) suspension: 1 mL of fresh anticoagulant rabbit blood was collected and centrifuged at 2,500 rpm for 5 min, a resulting supernatant was removed, and a resulting precipitate was washed 3 times with phosphate-buffered saline (PBS) and then suspended; and 500 μL of a resulting suspension was taken and added to a 50 mL centrifuge tube, and PBS was added to 50 mL.

Hemolysis experiment: Kaolin, flaky kaolinite, and tubular halloysite each were prepared with PBS into solutions with concentrations of 0.125 mg/mL, 0.25 mg/mL, 0.5 mg/mL, 1.0 mg/mL, and 2.0 mg/mL, respectively, which each were of 3 mL. 500 μL of the solution of each concentration was taken and thoroughly mixed with 500 μL of a prepared 2% RBC suspension. A positive control group was set as follows: 500 μL of deionized water was mixed with 500 μL of the 2% RBC suspension; and a negative control group was set as follows: 500 μL of PBS was mixed with 500 μL of the 2% RBC suspension. 3 replicates were set per group. A sample was incubated in a 37° C. water bath for 1 h and then centrifuged at 2,500 rpm, a resulting supernatant was collected, and the absorbance of the supernatant was measured by a microplate reader (414 nm).

Hemolysis rate (%)=(absorbance of a sample−absorbance of negative control)/(absorbance of positive control−absorbance of negative control)×100%.

The lower the hemolysis, the higher the biocompatibility. When a hemolysis rate is lower than 5%, it is considered that there is no hemolysis.

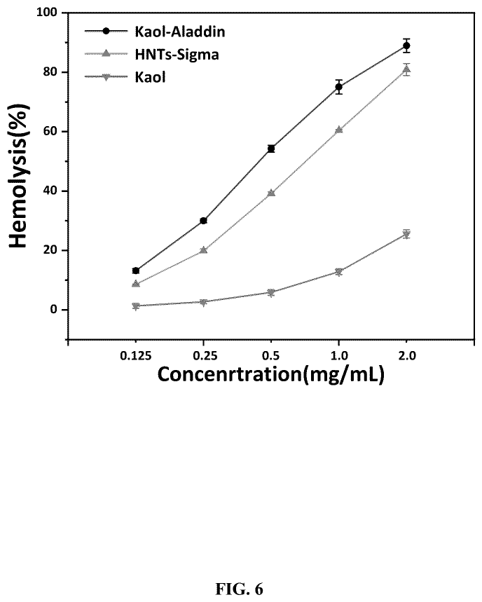

Hemolysis test results were shown in FIG. 6. It can be seen from FIG. 6 that Kaol leads to a lower hemolysis rate than Kaol-Aladdin and HNTs-Sigma, indicating that Kaol has optimal biosafety.

In Vitro Procoagulant Experiment:

100 μL of anticoagulant whole blood was added dropwise to a 6-well plate, and 10 μL of a 0.2 mol/L CaCl₂ solution was quickly added dropwise to the whole blood to recalcify the whole blood; 10 mg of a material was added to the whole blood by a dropper with a rubber head removed, and no material was added in a blank control group; the plate was incubated in a 37° C. water bath for 9 min, and then 10 mL of deionized water was slowly added dropwise around blood droplets, where coagulated blood was prevented from being impacted; and after the dropwise addition was completed, 1 mL of a resulting aqueous solution was immediately taken and centrifuged at 1,000 rpm, and then the absorbance (540 nm) was measured by a microplate reader.

10 mg of each of kaolin and the prepared different iron oxide/Kaol composite hemostatic materials was weighed to prepare experimental groups, and no material was added in the blank control group; and 3 replicates were set per group.

In vitro procoagulant test results were shown in FIG. 4. It can be seen from FIG. 4 that α-Fe₂O₃/Kaol has lower absorbance than Fe₃O₄/Kaol, γ-Fe₂O₃/Kaol, and FeOOH/Kaol, indicating that α-Fe₂O₃/Kaol has optimal procoagulant performance.

In Vitro Bleeding Time Experiment:

10 mg of each of Kaol, Ce-α-Fe₂O₃/Kaol-Aladdin, Ce-α-Fe₂O₃/HNTs-Sigma, α-Fe₂O₃/Kaol, and Ce-α-Fe₂O₃/Kaol composite hemostatic materials was weighed, added to a 2 mL centrifuge tube, and pre-warmed in a 37° C. water bath for 3 min; 200 μL of anticoagulant whole blood of a New Zealand white rabbit was added dropwise to a sample powder at a bottom of the centrifuge tube, and then 10 μL of a 0.2 mol/L CaCl₂) solution was quickly added dropwise to a resulting mixed system to calcify the blood to trigger blood coagulation; and a resulting mixed system was immediately incubated in a 37° C. water bath, during which the centrifuge tube was shaken every 15 s, the flow of blood in the tube was observed until the blood was coagulated, and a hemostasis time was recorded. 3 replicates were set for each material.

In vitro bleeding time test results were shown in FIG. 5. It can be seen from FIG. 5 that, compared with the α-Fe₂O₃/Kaol (Example 6), a bleeding time of the Ce-α-Fe₂O₃/Kaol composite hemostatic material (Example 7) is shortened, indicating that the Ce doping can improve a hemostatic effect of the composite hemostatic material.

In Vivo Hemostasis Experiment:

6-week-old female Kunming mice were selected and randomly grouped according to a body weight, with 5 mice in each group. Each mouse was fixed with a tail exposed, and a 1 cm wound was cut with a scalpel blade on a tail vein of the mouse to allow bleeding. Then a corresponding material powder was quickly applied to the wound, the wound was immediately covered with a hemostatic gauze and gently pressed to stop the bleeding until the bleeding was completely stopped, and a hemostasis time was recorded by a timer. Blood flowing out from the wound was dipped by a gauze and weighed, and a bleeding amount was calculated. Hemostasis time and bleeding amount results were shown in Table 2.

TABLE 2
Bleeding times and bleeding amounts of Ce-α-Fe₂O₃/Kaol
composite hemostatic materials

| | | Hemostasis | Bleeding |
|---|---|---|---|
| Group | Material | time (s) | amount (mg) |
| Blank control | No material is added | 179.8 ± 36.6 | 132.7 ± 75.9 |
| group | | | |
| Raw ore | Kaol | 153.2 ± 33.7 | 104.8 ± 50.5 |
| Example 6 | α-Fe₂O₃/Kaol | 136 ± 34.7 | 96.4 ± 42.6 |
| Example 7 | Ce-α-Fe₂O₃/Kaol | 112.6 ± 23.4 | 84.81 ± 40.4 |
| Positive | Yunnan Baiyao | 136.6 ± 40.9 | 53.1 ± 32.5 |
| control group | | | |

It can be seen from Table 2 that the doping of Ce in α-Fe₂O₃/Kaol can effectively improve a hemostasis rate and reduce a bleeding amount.

### 6 Gauze Products with Different Loads Obtained Through Impregnation with Hemostatic Material Suspensions of Different Concentrations (Examples 10 to 15)

### Preparation of a Ce-α-Fe₂O₃/Kaol Composite Hemostatic Agent

5 g of kaolin and 250 mL of the polyhydroxyferric ion solution were mixed and stirred, 20 mL of the Ce(NO₃)₃ solution was added dropwise, and a pH of a resulting mixed solution was adjusted with a 5 mol/L NaOH solution to about 3; a resulting system was stirred at 60° C. for 5 h and then centrifuged at 8,000 rpm, and a resulting precipitate was washed 3 times and dried to obtain a Ce—FeOOH/Kaol composite. The Ce—FeOOH/Kaol composite was ground and then calcined (including calcining at 250° C. for 1 h, calcining at 350° C. for 1 h, and calcining at 550° C. for 4 h) to obtain the Ce-α-Fe₂O₃/Kaol composite hemostatic material.

The Ce-α-Fe₂O₃/Kaol composite hemostatic material prepared above was adopted in each of Examples 10 to 15.

### Example 10

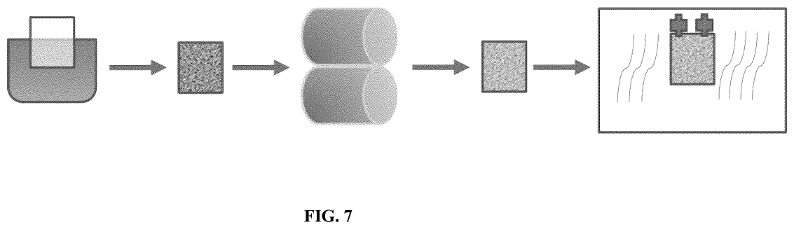

As shown in FIG. 7, 0.2 g of the Ce-α-Fe₂O₃/Kaol hemostatic material was added to 200 mL of water, and a resulting mixture was thoroughly stirred to obtain a homogeneous suspension; a piece of a non-woven fabric (area: 10*9.5 cm²) was cut and directly impregnated with the homogeneous suspension such that the material adhered to a surface of the non-woven fabric, where upper and lower sides of the non-woven fabric each were impregnated once, with a total impregnation time of about 2 s; an impregnated non-woven fabric was placed on a rolling machine and pressed once with a distance of 0.05 mm between upper and lower rollers of the rolling machine to strengthen an adhesion degree between the powdery material and the non-woven fabric; and finally, the pressed non-woven fabric was hung by dovetail clips in an oven at 60° C. and blow-dried.

### Example 11

0.5 g of the Ce-α-Fe₂O₃/Kaol hemostatic material was added to 200 mL of water, and a resulting mixture was thoroughly stirred to obtain a homogeneous suspension; a piece of a non-woven fabric (area: 10*9.5 cm²) was cut and directly impregnated with the homogeneous suspension such that the material adhered to a surface of the non-woven fabric, where upper and lower sides of the non-woven fabric each were impregnated once, with a total impregnation time of about 2 s; an impregnated non-woven fabric was placed on a rolling machine and pressed once with a distance of 0.05 mm between upper and lower rollers of the rolling machine to strengthen an adhesion degree between the powdery material and the non-woven fabric; and finally, the pressed non-woven fabric was hung by dovetail clips in an oven at 60° C. and blow-dried.

### Example 12

1.0 g of the Ce-α-Fe₂O₃/Kaol hemostatic material was added to 200 mL of water, and a resulting mixture was thoroughly stirred to obtain a homogeneous suspension; a piece of a non-woven fabric (area: 10*9.5 cm²) was cut and directly impregnated with the homogeneous suspension such that the material adhered to a surface of the non-woven fabric, where upper and lower sides of the non-woven fabric each were impregnated once, with a total impregnation time of about 2 s; an impregnated non-woven fabric was placed on a rolling machine and pressed once with a distance of 0.05 mm between upper and lower rollers of the rolling machine to strengthen an adhesion degree between the powdery material and the non-woven fabric; and finally, the pressed non-woven fabric was hung by dovetail clips in an oven at 60° C. and blow-dried.

### Example 13

2.0 g of the Ce-α-Fe₂O₃/Kaol hemostatic material was added to 200 mL of water, and a resulting mixture was thoroughly stirred to obtain a homogeneous suspension; a piece of a non-woven fabric (area: 10*9.5 cm²) was cut and directly impregnated with the homogeneous suspension such that the material adhered to a surface of the non-woven fabric, where upper and lower sides of the non-woven fabric each were impregnated once, with a total impregnation time of about 2 s; an impregnated non-woven fabric was placed on a rolling machine and pressed once with a distance of 0.05 mm between upper and lower rollers of the rolling machine to strengthen an adhesion degree between the powdery material and the non-woven fabric; and finally, the pressed non-woven fabric was hung by dovetail clips in an oven at 60° C. and blow-dried.

### Example 14

5.0 g of the Ce-α-Fe₂O₃/Kaol hemostatic material was added to 200 mL of water, and a resulting mixture was thoroughly stirred to obtain a homogeneous suspension; a piece of a non-woven fabric (area: 10*9.5 cm²) was cut and directly impregnated with the homogeneous suspension such that the material adhered to a surface of the non-woven fabric, where upper and lower sides of the non-woven fabric each were impregnated once, with a total impregnation time of about 2 s; an impregnated non-woven fabric was placed on a rolling machine and pressed once with a distance of 0.05 mm between upper and lower rollers of the rolling machine to strengthen an adhesion degree between the powdery material and the non-woven fabric; and finally, the pressed non-woven fabric was hung by dovetail clips in an oven at 60° C. and blow-dried.

### Example 15

10.0 g of the Ce-α-Fe₂O₃/Kaol hemostatic material was added to 200 mL of water, and a resulting mixture was thoroughly stirred to obtain a homogeneous suspension; a piece of a non-woven fabric (area: 10*9.5 cm²) was cut and directly impregnated with the homogeneous suspension such that the material adhered to a surface of the non-woven fabric, where upper and lower sides of the non-woven fabric each were impregnated once, with a total impregnation time of about 2 s; an impregnated non-woven fabric was placed on a rolling machine and pressed once with a distance of 0.05 mm between upper and lower rollers of the rolling machine to strengthen an adhesion degree between the powdery material and the non-woven fabric; and finally, the pressed non-woven fabric was hung by dovetail clips in an oven at 60° C. and blow-dried.

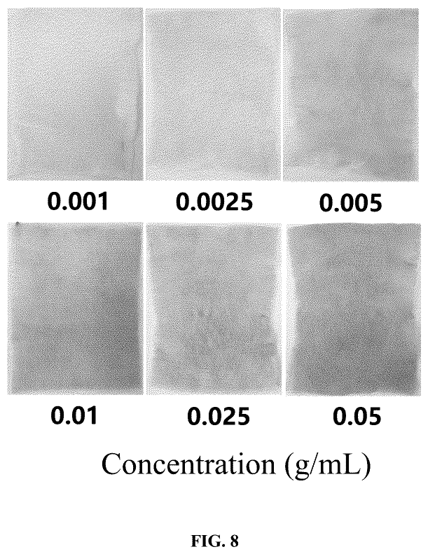

FIG. 8 shows pictures of 6 gauze products with different loads obtained through impregnation with hemostatic material suspensions of different concentrations; and as shown in FIG. 8, the Ce-α-Fe₂O₃/Kaol hemostatic material is successfully loaded on the gauze.

Loads of the hemostatic materials in the gauze products obtained in Examples 10 to 15 were shown in Table 3.

TABLE 3
Loads of hemostatic materials in Ce-α-Fe₂O₃/Kaol gauze products

| | Mass of a powder | | | | | |
|---|---|---|---|---|---|---|
| | in 200 mL of | Concentration | Mass of a nude | Mass after | Load | Load |
| Example | water (g) | (g/mL) | fabric (g) | oven-drying (g) | (g/cm²) | (g) |
| Example 10 | 0.2 | 0.0010 | 0.5039 | 0.5056 | 0.000000000 | 0.0017 |
| Example 11 | 0.5 | 0.0025 | 0.5165 | 0.5310 | 0.000152632 | 0.0145 |
| Example 12 | 1.0 | 0.0050 | 0.5068 | 0.5517 | 0.000472632 | 0.0449 |
| Example 13 | 2.0 | 0.0100 | 0.4966 | 0.5756 | 0.000831579 | 0.0790 |
| Example 14 | 5.0 | 0.0250 | 0.5016 | 0.7448 | 0.002560000 | 0.2432 |
| Example 15 | 10.0 | 0.0500 | 0.5188 | 0.8391 | 0.003371579 | 0.3203 |

In Vivo Hemostasis Experiment:

Liver Hemostasis Experiment:

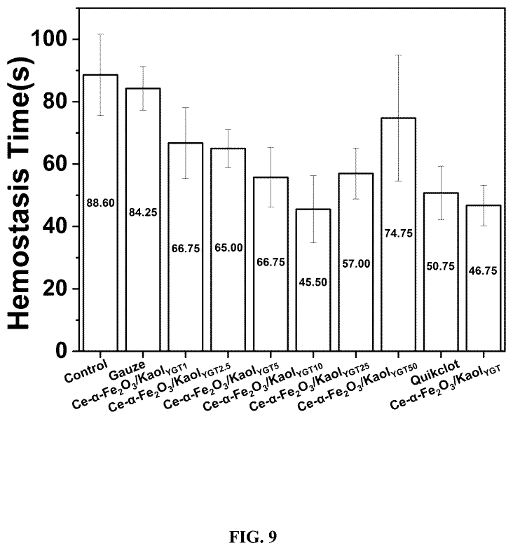

6-week-old female Kunming mice were selected and randomly grouped according to a body weight, with 5 mice in each group. Each mouse was anesthetized and fixed, an abdominal cavity of the mouse was opened, and a wound of about 1 cm was cut with a scalpel on a left lobe tissue of the liver; the bleeding left lobe of the liver was covered with a gauze sample (area: 47.5 cm²) (or a powdery sample); and a hemostasis time was recorded, and a mass change of the gauze sample was measured to calculate a bleeding amount. Hemostasis time and bleeding amount results were shown in Table 4 and FIG. 9.

TABLE 4
Bleeding times and bleeding amounts of Ce-α-Fe₂O₃/
Kaol hemostatic gauzes

| Group | Material | Hemostasis time (s) | Bleeding amount (g) |
|---|---|---|---|
| Control group | No material is added | 88.60 ± 13.00 | 0.2686 ± 0.1892 |
| Blank gauze | Additive-free gauze | 84.25 ± 7.01 | 0.1262 ± 0.0506 |
| Example 10 | Ce-α-Fe₂O₃/Kaol_{YGT1}-loaded gauze | 66.75 ± 11.34 | 0.1711 ± 0.0377 |
| Example 11 | Ce-α-Fe₂O₃/Kaol_{YGT2.5}-loaded gauze | 65.00 ± 6.16 | 0.1038 ± 0.0215 |
| Example 12 | Ce-α-Fe₂O₃/Kaol_{YGT5}-loaded gauze | 55.75 ± 9.62 | 0.1522 ± 0.0793 |
| Example 13 | Ce-α-Fe₂O₃/Kaol_{YGT10}-loaded gauze | 45.50 ± 10.78 | 0.1161 ± 0.0353 |
| Example 14 | Ce-α-Fe₂O₃/Kaol_{YGT25}-loaded gauze | 57.00 ± 8.15 | 0.2188 ± 0.0884 |
| Example 15 | Ce-α-Fe₂O₃/Kaol_{YGT50}-loaded gauze | 74.75 ± 20.11 | 0.1998 ± 0.1214 |
| Quikclot | Quikclot gauze | 50.75 ± 8.58 | 0.2266 ± 0.1769 |
| Powdery sample | Ce-α-Fe₂O₃/Kaol_{YGT} | 46.75 ± 6.49 | 0.0733 ± 0.0338 |

Tail Vein Hemostasis Experiment:

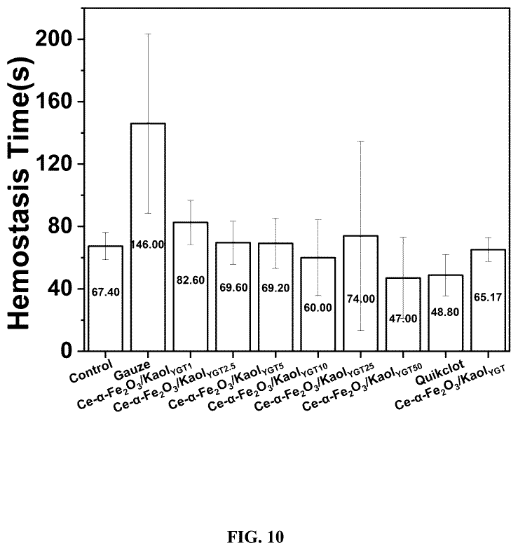

6-week-old female Kunming mice were selected and randomly grouped according to a body weight, with 5 mice in each group. Each mouse was fixed with a tail exposed, and a 1 cm wound was cut with a scalpel blade on a tail vein of the mouse to allow bleeding; then a corresponding material powder was quickly applied to the wound, the wound was immediately covered with a hemostatic gauze and gently pressed to stop the bleeding until the bleeding was completely stopped, and a hemostasis time was recorded by a timer; and blood flowing out from the wound was dipped by a gauze and weighed, and a bleeding amount was calculated. Hemostasis time and bleeding amount results were shown in Table 5 and FIG. 10.

TABLE 5
Bleeding times and bleeding amounts of Ce-α-Fe₂O₃/
Kaol hemostatic gauzes

| Group | Material | Hemostasis time (s) | Bleeding amount (g) |
|---|---|---|---|
| Control group | No material is added | 67.40 ± 8.8 | 0.00934 ± 0.00588 |
| Blank gauze | Additive-free gauze | 146.00 ± 57.5 | 0.03402 ± 0.02575 |
| Example 10 | Ce-α-Fe₂O₃/Kaol_{YGT1}-loaded gauze | 82.60 ± 14.2 | 0.03584 ± 0.02089 |
| Example 11 | Ce-α-Fe₂O₃/Kaol_{YGT2.5}-loaded gauze | 69.60 ± 13.9 | 0.03970 ± 0.01297 |
| Example 12 | Ce-α-Fe₂O₃/Kaol_{YGT5}-loaded gauze | 69.20 ± 16.12 | 0.02690 ± 0.01991 |
| Example 13 | Ce-α-Fe₂O₃/Kaol_{YGT10}-loaded gauze | 60.00 ± 24.37 | 0.01326 ± 0.00807 |
| Example 14 | Ce-α-Fe₂O₃/Kaol_{YGT25}-loaded gauze | 74.00 ± 60.64 | 0.03230 ± 0.01342 |
| Example 15 | Ce-α-Fe₂O₃/Kaol_{YGT50}-loaded gauze | 47.00 ± 26.12 | 0.01500 ± 0.01487 |
| Quikclot | Quikclot gauze | 48.80 ± 13.30 | 0.00806 ± 0.00560 |
| Powdery sample | Ce-α-Fe₂O₃/Kaol_{YGT} | 65.17 ± 7.62 | 0.01737 ± 0.00870 |

It can be seen from Table 4, Table 5, FIG. 9, and FIG. 10 that, compared with the existing hemostatic gauzes on the market, the Ce-α-Fe₂O₃/Kaol hemostatic gauze can improve a hemostasis rate and reduce a bleeding amount to some degree.

What is not mentioned above can be acquired in the prior art.

Although some specific embodiments of the present disclosure have been described in detail by way of examples, those skilled in the art will appreciate that the above examples are provided for illustration only and not for limiting the scope of the present disclosure. A person skilled in the art can make various modifications or supplements to the specific embodiments described or replace them in a similar manner, but it may not depart from the direction of the present disclosure or the scope defined by the appended claims. Those skilled in the art should understand that any modification, equivalent replacement, and improvement that are made to the above embodiments according to the technical essence of the present disclosure shall be included in the protection scope of the present disclosure.

1. A kaolin-based hemostatic gauze, comprising:
a medical non-woven fabric as a carrier; and
a kaolin-containing composite hemostatic material loaded on the medical non-woven fabric;
wherein kaolin serves as a carrier in the kaolin-containing composite hemostatic material, the Kaolin is doped with Ce, and α-Fe₂O₃ is loaded on the Kaolin; the kaolin comprises flaky kaolinite and tubular halloysite;
the kaolin-containing composite hemostatic material is prepared according to the following steps:
S1: mechanically mixing the kaolin with a polyhydroxyferric ion solution and a Ce salt solution; and
S2: calcining a resulting mixture at a specified temperature to obtain a hemostatic material α-Fe₂O₃/Kaol with high biocompatibility, wherein the hemostatic material α-Fe₂O₃/Kaol is the kaolin-containing composite hemostatic material.

2. (canceled)

3. The kaolin-based hemostatic gauze according to claim 1, wherein the kaolin is a raw ore.

4. The kaolin-based hemostatic gauze according to claim 1, wherein a loading rate of the α-Fe₂O₃ is 50% to 70%.

5. (canceled)

6. The kaolin-based hemostatic gauze according to claim 1, wherein the specified temperature is 500° C. to 600° C.

7. The kaolin-based hemostatic gauze according to claim 1, wherein the specified temperature is 550° C.

8. The kaolin-based hemostatic gauze according to claim 1, wherein the polyhydroxyferric ion solution has a concentration of 0.4 mol/L, and a mass-to-volume ratio of the kaolin to the polyhydroxyferric ion solution is 1:50 g/mL; the Ce salt solution is a Ce(NO₃)₃ solution; and the Ce(NO₃)₃ solution has a concentration of 0.1 mol/L to 1.0 mol/L.

9. A method for preparing the kaolin-based hemostatic gauze according to claim 1, comprising the following steps:
S1: preparing the kaolin-containing composite hemostatic material;
S2: preparing the kaolin-containing composite hemostatic material into a suspension;
S3: impregnating the medical non-woven fabric with the suspension thoroughly stirred, wherein upper and lower sides of the medical non-woven fabric each are impregnated once; and
S4: pressing and oven-drying an impregnated medical non-woven fabric to obtain the kaolin-based hemostatic gauze.

10. The method according to claim 9, wherein the suspension of the kaolin-containing composite hemostatic material has a concentration of 0.001 g/mL to 0.05 g/mL.
